(19) United States
(12) Patent Application Publication
(10) Pub. No.: US 2017/0176468 A1
(43) Pub. Date: Jun. 22, 2017

(54) METHOD FOR DETERMINATION OF VITELLOGENIN AS BIOMARKER FOR AN EXOGENOUS OESTROGENIC EFFECT ON FISH

(71) Applicants: Gobio GmbH, Aarbergen/Kettenbach (DE); TECO MEDICAL AG, Sissach (CH)

(72) Inventors: Bernhard Allner, Aarbergen/Kettenbach (DE); Mark Hennies, Sissach (CH)

(21) Appl. No.: 15/309,600

(22) PCT Filed: May 10, 2014
(86) PCT No.: PCT/DE2014/100161

§ 371 Date: Jan. 10, 2017

(51) Int. Cl. G01N 33/74; G01N 1/28

(52) U.S. Cl. CPC G01N 33/743; G01N 1/286; G01N 2333/4603

(57) ABSTRACT

The invention relates to a method for the determination of vitellogenin as a biomarker or end point for an exogenous oestrogenic effect on fish. The problem addressed by the invention is the creation of a non-destructive method in which the laboratory animals do not have to be killed. The method also should not necessitate any additional use of laboratory animals for carrying out tests on oestrogens, anti-oestrogens, and endocrine disruptors. This problem is solved by a method for the determination of vitellogenin as biomarker for an exogenous oestrogenic effect on fish comprising the method steps of taking a skin mucus sample from a fish by swabbing, transferring the skin mucus sample to a reaction vessel, homogenising the skin mucus sample, and taking an aliquot of the skin mucus sample for vitellogenin determination by means of an ELISA method of detection.

Regular testing of the effect of chemicals present in the environment on living organisms, especially on vertebrates, is necessary. This can involve testing, inter alia, whether chemicals and/or their degradation products have, for example, an effect on the endocrine system.

In this connection, numerous investigations have already shown that especially estrogen effects are mimicked owing to an interaction of chemicals with the estrogen receptors in vertebrates, in particular in fishes, leading to, for example, reproduction disorders in male fishes following exposure to wastewaters.

As part of this environmental monitoring, which is done according to exact requirements of OECD test guidelines, an immunological recording of vitellogenin by means of antibodies is currently carried out according to established assay methods, the paradoxical induction of yolk proteins (vitellogenin) being determined from total body homogenates or in the blood serum/plasma of test fishes. This method is, for example, described in the final report, BMBF [Federal Ministry of Education and Research] funding number 02WA9981/9, of the Hessisches Landesamt für Umwelt and Geologie [Hessian state office for the environment and geology], February 2003.

In egg-laying animals, vitellogenin is an egg yolk precursor protein which is formed in the liver owing to estrogenic induction during the main phase of oocyte growth. This yolk protein migrates in the bloodstream of female fishes to the ovaries, where it breaks down into other yolk proteins, which later serve as a food reserve for the developing fish embryos in the egg. If there is now an artificially induced synthesis of vitellogenin in male and young fishes owing to xenoestrogens in waters, for example owing to contamination of the water with sludges, then the consequence is reproduction disorders in these fishes.

With the aid of the currently used vitellogenin assays, it is possible to determine in fishes the estrogen status, the status of sexual development, the identification of genders if there is no sexual dimorphism; it is possible to conduct the detection of an estrogenic effect in the case of natural or anthropogenic substances or chemicals and to test waters or wastewaters for estrogen-effective substances.

However, the currently known assay methods are destructive for the test fishes usable in chemical testing or in environmental monitoring.

Also, the conventional assays invariably require a separate procedure and thus necessitate an increased animal usage in chemical testing.

Moreover, the desired paradoxical induction of vitellogenin can, after relatively long exposure, be inhibited via feedback mechanisms, and so the conventional assays are less sensitive.

Furthermore, owing to the destructiveness of the conventional methods, the recording of estrogenic efficacy is only possible by means of a vitellogenin determination at the end of the exposure time of a sublethal long-term test. This complicates the coupling of long-term tests for recording general chronic toxicity with the determination of this effect-specific end point.

Likewise in the currently used assay methods, a disadvantage is that the matrix, from which the determination is carried out, can be defined only with difficulty and is subject to large variations. Specifically when collecting blood samples, there is frequently contamination by lymph.

Also, sample preparation, such as obtaining serum or preparing suitable homogenates, is currently highly complicated and barely standardizable.

Vitellogenin is a highly unstable protein. Both the protein as target analyte in its biological matrix (in the sample) and standard solutions containing defined protein amounts break down. Therefore, conventional assays are not suitable for an absolute quantification, since fragments are also immunologically recorded.

Lastly, the known conventional assays are generally only applicable to a small number of very closely related species.

Against this background, the object of the disclosed inventive method for determining vitellogenin as biomarker or end point for an exogenous estrogenic effect on fishes is to provide a nondestructive method in which the test animals need not be killed. It is also intended here that the method require no additional usage of test animals for carrying out tests for estrogenic, antiestrogenic and endocrine disruptors.

The method according to the invention for determining vitellogenin as biomarker or end point for an exogenous estrogenic effect on fishes comprises the method steps of
a) taking a skin mucus sample of a fish by swabbing,
b) transferring the skin mucus sample to a reaction vessel,
c) homogenizing the skin mucus sample,
d) removing an aliquot of the skin mucus sample for vitellogenin determination by means of an ELISA detection method.

Therefore, the method is a nondestructive method in which the test animals need not be killed and also no additional usage of test animals for carrying out tests for estrogenic, antiestrogenic and endocrine disruptors is required.

After an exposure time of 4-7 days, it is possible to record the efficacy of the exogenous noxious agents (harmful substances) at the time of maximal induction of vitellogenin and hence of optimal species-specific sensitivity.

Estrogenic efficacy can be tested as an additional end point, i.e., coupled to subchronic long-term tests such as OECD 215, or in sublethal concentrations of the acute test OECD 203. No additional experimental effort is required therefore.

The matrix disclosed in the disclosed method according to the invention is well defined in contrast to conventional assays. The samples do not need to be separated from non-target tissues such as blood, lymph, epithelia and others.

Also, complicated sample preparation procedures are not necessary.

Appropriately, in an expansion of the method, a further aliquot of the homogenized skin mucus sample is removed as parallel sample and a further vitellogenin determination by means of an ELISA detection method is carried out. In this way, the first detection is checked on the basis of said parallel sample and a greater reliability is achieved with regard to the results of the detection method.

In an advantageous expansion of the method, a further aliquot of the homogenized skin mucus sample can be removed and a total protein determination can be carried out, since this makes possible a determination of the relationship of the total protein content to the previously determined vitellogenin content of the skin mucus sample, which relationship gives further information. Here, said total protein determination can be carried out both in a method as claimed in the main claim and in a method in combination with the parallel sample as claimed in claim 2.

The skin mucus sample of the fish can be usefully taken here by means of a forensic swab with predetermined breaking point. Here, the use of the swab with predetermined breaking point has advantages especially in combination with a method workflow in which the forensic swab remains as agitator in the reaction vessel during homogenization of the skin mucus sample. A micro-reaction vessel can be appropriately used here.

Moreover, it has been found to be very advantageous for the method when the skin mucus sample is transferred to a reaction vessel containing protein-stabilizing buffer. The use of suitable buffers prevents the protein from breaking down, making it possible to apply the assay to a larger number of species (taxonomic groups) such as Cypriniformes fishes for example.

A further appropriate aspect of the method is that the skin mucus sample is taken on the dorsal body region of the fish. It has been found that said body region of the fish is particularly suited for the removal of the skin mucus sample.

To carry out the detection method after the homogenization of the sample, it has been found to be advantageous to transfer the aliquot to a microtiter plate for further determination. A standardized evaluation in the laboratory is thus easily possible. This applies especially to the discussed ELISA method as antibody-based detection method or immunoassay method that is based on an enzymatic color reaction.

The method according to the invention has been tested and is especially suitable for Cypriniformes fishes, for Perciformes fishes and for Salmoniformes fishes, and in the case of the Cypriniformes fishes those of the species common carp (Cyprinus carpio), goldfish (Carassius auratus), roach (Rutilus rutilus), zebrafish (Danio rerio), fathead minnow (Pimephales promelas) or Japanese rice fish (Oryzias latipes) are disclosed.

In the case of the Perciformes fishes, fishes of the species European perch (Perca fluviatilis), Nile tilapia (Oreochromis niloticus), three-spined stickleback (Gasterosteus aculeatus) or Australian rainbow fishes (Melanotaenia species) were advantageously used, and in the case of the Salmoniformes fishes, those of the species brown trout (Salmo trutta forma fario), rainbow trout (Oncorhynchus mykiss) or Arctic char (Salvelinus alpinus) were advantageously used as suitable test animals.

The disclosed method according to the invention can be used, inter alia, for the detection of estrogenic effects in laboratory experiments as an additional end point in standard methods, for determining the spawning maturity of female Cypriniformes fishes and for recording the exposure to estrogenically effective noxious environmental agents outdoors. It is also easily possible with this method to record a disturbance in sexual maturity due to antiestrogenic noxious environmental agents outdoors.

1. A method for determining vitellogenin as biomarker for an exogenous estrogenic effect on fishes, the method comprising:
a) taking a skin mucus sample of a fish by swabbing,
b) transferring the skin mucus sample to a reaction vessel,
c) homogenizing the skin mucus sample, and
d) removing an aliquot of the skin mucus sample for vitellogenin determination by means of an ELISA detection method.

2. The method as claimed in claim 1,
wherein,
a further aliquot of the homogenized skin mucus sample is removed as parallel sample and a further vitellogenin determination by means of an ELISA detection method is carried out.

3. The method as claimed in claim 1,
wherein,
a further aliquot of the homogenized skin mucus sample is removed and a total protein determination is carried out in order to determine the relationship of the total protein content to the vitellogenin content of the skin mucus sample.

4. The method as claimed in claim 1,
wherein,
the skin mucus sample of the fish is taken by means of a forensic swab with predetermined breaking point.

5. The method as claimed in claim 1,
wherein,
the skin mucus sample is transferred to a micro-reaction vessel containing protein-stabilizing buffer.

6. The method as claimed in claim 4,
wherein,
the forensic swab remains as agitator in the micro-reaction vessel during homogenization of the skin mucus sample.

7. The method as claimed in claim 1,
wherein,
the skin mucus sample is taken on the dorsal body region of the fish.

8. The method as claimed in claim 1,
wherein,
the aliquot is transferred to a microtiter plate for further determination.

9. The method as claimed in claim 1,
wherein,
the fishes are Cypriniformes fishes.

10. The method as claimed in claim 1,
wherein,
the fishes are Perciformes fishes.

11. The method as claimed in claim 1,
wherein,
the fishes are Salmoniformes fishes.

12. The method as claimed in claim 9,
wherein,
the Cypriniformes fishes originate from the species common carp (Cyprinus carpio), goldfish (Carassius auratus), roach (Rutilus rutilus), zebrafish (Danio rerio), fathead minnow (Pimephales promelas) or Japanese rice fish (Oryzias latipes).

13. The method as claimed in claim 10,
wherein,
the Perciformes fishes originate from the species European perch (Perca fluviatilis), Nile tilapia (Oreochromis niloticus), three-spined stickleback (Gasterosteus aculeatus) or Australian rainbow fishes (Melanotaenia species).

14. The method as claimed in claim 11,
wherein,
the Salmoniformes fishes originate from the species brown trout (Salmo trutta forma fario), rainbow trout (Oncorhynchus mykiss) or Arctic char (Salvelinus alpinus).

15. The method as claimed in claim 1,
wherein,
a micro-reaction vessel is used as reaction vessel.
